(19) United States
(12) Patent Application Publication
(10) Pub. No.: US 2026/0338744 A1
(43) Pub. Date: Oct. 29, 2026

(54) METHOD FOR DETECTING SEX OF HATCHING EGGS IN EARLY STAGE OF INCUBATION

(71) Applicant: ZHEJIANG UNIVERSITY, Zhejiang (CN)

(72) Inventors: Jinming PAN, Zhejiang (CN); Shaomin XU, Zhejiang (CN); Lijuan XIE, Zhejiang (CN); Yibin YING, Zhejiang (CN); Hongjian LIN, Zhejiang (CN); Jintian CHEN, Zhejiang (CN); Mengzui DI, Zhejiang (CN)

(73) Assignee: ZHEJIANG UNIVERSITY, Zhejiang (CN)

(21) Appl. No.: 19/550,303

(22) Filed: Feb. 26, 2026

Related U.S. Application Data
(63) Continuation of application No. PCT/CN2025/088685, filed on Apr. 14, 2025.

(30) Foreign Application Priority Data
Mar. 19, 2025 (CN) 202510326934.2

(51) Int. Cl. G01N 30/72; A01K 43/00; G01N 1/28; G01N 30/02; G01N 30/86; G16B 40/10; G16B 40/20

(52) U.S. Cl. CPC G01N 30/7233; A01K 43/00; G01N 1/286; G01N 30/8686; G16B 40/10; G16B 40/20; G01N 2001/2886; G01N 2030/027

(57) ABSTRACT

Disclosed is a method for detecting sex of hatching eggs in an early stage of incubation. The method includes: extracting allantoic fluid of hatching eggs in early stage of incubation through an extraction device; obtaining allantoic fluid metabolite characteristics after performing a metabolomics analysis on the allantoic fluid, performing a bioinformatics analysis combined with in vitro cell culture to confirm sex-specific metabolites; further performing fractionation processing on the allantoic fluid to obtain low-molecular-weight allantoic fluid containing the sex-specific metabolites; performing a spectral comparative analysis on the allantoic fluid to be detected, thereby constructing a multi-machine learning allantoic fluid sex classification model. The model enables rapid sex determination of hatching eggs during the early incubation stage, achieving a balance between the time window, accuracy, and cost control.

### CROSS-REFERENCE TO RELATED APPLICATION

This application is a continuation of international PCT application serial no. PCT/CN2025/088685, filed on Apr. 14, 2025, which claims the priority benefit of China application serial no. 202510326934.2, filed on Mar. 19, 2025. The entirety of each of the above-mentioned patent applications is hereby incorporated by reference herein and made a part of this specification.

### TECHNICAL FIELD

The present disclosure relates to a method for determining the sex of chicken embryos, pertaining to the technical field of agricultural and livestock product testing, and specifically involving a method for detecting sex of hatching eggs in an early stage of incubation.

### DESCRIPTION OF RELATED ART

Eggs are a significant agricultural product, serving as a daily staple food, a source of nutrition, and a primary source of protein. Egg production has consistently accounted for a major proportion of poultry egg production. However, a critical issue confronting the egg-laying industry is that a number of young male chickens are prematurely culled each year due to their inability to lay eggs and their limited market viability, leading to resource wastage and animal welfare concerns. The core focus of research is to achieve early, precise, and rapid sex detection of chicken embryos. Allantoic fluid, as an exogenous sample of the embryos, offers advantages such as relatively easy sampling, low invasiveness, and high hatchability. Currently, researchers extract allantoic fluid from fertilized eggs between the 7th and 10th day of incubation to measure the concentration of estrone sulfate in the fluid. This is determined using the Enzyme-Linked Immunosorbent Assay (ELISA) method to ascertain the concentration of estrone sulfate, which is subsequently used to determine the sex of the chicken embryos within the fertilized eggs. Additionally, some researchers have designed methods for detecting sex of chicken eggs, which include the collection, transportation, and pre-treatment of allantoic fluid of chicken embryos, as well as the detection of estrogen concentration within the allantoic fluid. This research provides valuable information for understanding sex detection in chicken embryos. However, the composition of allantoic fluid is complex and dynamic, and the basis of existing research on allantoic fluid is relatively limited. This is particularly challenging in terms of balancing cost, rapid detection, and the need for low invasiveness. Therefore, it is of significant importance to explore the sex-related differences in the allantoic fluid and construct an efficient and accurate rapid detection model based on this information.

### SUMMARY

To address the issues present in the background technology, the present disclosure provides a method for detecting sex of hatching eggs in an early stage of incubation. This method involves a metabolomics analysis of allantoic fluid to obtain characteristic metabolites of the fluid. A bioinformatics analysis combined with in vitro cell culture is used to confirm sex-specific metabolites. The allantoic fluid is then subjected to concentration and fractionation processes to obtain low-molecular-weight allantoic fluid containing sex-specific metabolites. A spectral comparative analysis is conducted on the multi-component allantoic fluid samples to construct a multi-component, multiple machine learning models for sex classification. This enables rapid classification of the sex of chicken embryos.

The technical solution adopted by the present disclosure is:

The method for detecting sex of hatching eggs in the early stage of incubation of the present disclosure includes:
Step 1: setting up an extraction device, and extracting allantoic fluid from a plurality of hatching eggs of known sex respectively in the early stage of incubation through the extraction device.
Step 2: performing a metabolomics analysis and a bioinformatics analysis on the allantoic fluid of each of the hatching eggs sequentially to confirm sex-specific metabolites.
Step 3: performing fractionation processing on the allantoic fluid of each of the hatching eggs to obtain the allantoic fluid containing the sex-specific metabolites.
Step 4: obtaining fingerprint spectral characteristic data of the allantoic fluid containing the sex-specific metabolites from each of the hatching eggs and adding labels of the respective known sex, thereby constructing a training set to input into multiple machine learning models for training as well as testing and validation, so as to obtain a trained multi-machine learning allantoic fluid sex classification model.
Step 5: extracting the allantoic fluid of the hatching eggs to be detected in the early stage of incubation through the extraction device, then obtaining the fingerprint spectral characteristic data of the allantoic fluid containing the sex-specific metabolites to be detected after performing the same processing as in steps 3 and 4, and inputting the fingerprint spectral characteristic data into the multi-machine learning allantoic fluid sex classification model, obtaining the sex of the hatching eggs to be detected in the early stage of incubation after processing, thus achieving rapid sex detection of the hatching eggs in the early stage of incubation.

In step 1, the extraction device includes a light source, an extractor, a laser perforator and an egg tray. After cleaning the hatching eggs in the early stage of incubation, the hatching eggs are placed vertically on the egg tray. The light source is placed on eggshells of top surfaces of the hatching eggs in the early stage of incubation and faces the interior of the hatching eggs in the early stage of incubation downward vertically to visualize positions of the allantoic fluid, chicken embryos, blood vessels, yolk and air cell inside the hatching eggs in the early stage of incubation, so as to avoid other parts and facilitate the extraction of the allantoic fluid. During the extraction of the allantoic fluid, the hatching eggs in the early stage of incubation are in a dark environment. The position of the allantoic fluid of the hatching eggs in the early stage of incubation is visualized through the light source, then laser perforation is performed on the eggshells of the hatching eggs in the early stage of incubation through the laser perforator to expose the position of the allantoic fluid, and finally the allantoic fluid is extracted from the hatching eggs through the extractor. Finally, liquid wax is utilized to seal a micropore to ensure normal incubation of the chicken embryos.

In the step 1, the early stage of incubation specifically refers to a period of 5 to 10 days of incubating the hatching eggs.

In the step 2, the metabolomics analysis is performed on the allantoic fluid of each of the hatching eggs. Firstly, the respective metabolites present in the allantoic fluid of each of the hatching eggs with the known sex are identified using Liquid Chromatography-Tandem Mass Spectrometry (LC-MS/MS) in the metabolomics analysis. Subsequently, a bioinformatics analysis method is employed to screen the sex-specific metabolites from the respective metabolites identified in the allantoic fluid of the hatching eggs with known sex.

In the step 2, the sex-specific metabolites include L-ornithine, proline and cytidine, etc.

In the step 3, the allantoic fluid of the hatching eggs to be detected is first concentrated and then fractionated according to molecular weight to obtain high-molecular-weight allantoic fluid, medium-molecular-weight allantoic fluid, and low-molecular-weight allantoic fluid. The low-molecular-weight allantoic fluid, which contains sex-specific metabolites, is used to enrich the metabolite information of the low-molecular-weight allantoic fluid.

In the step 4, for each of the hatching eggs, the allantoic fluid containing the sex-specific metabolites is enriched on a surface of a crystal or a slide. Subsequently, a spectral scan is conducted to collect data, thereby obtaining the fingerprint spectral characteristic data of the allantoic fluid.

The method designed by the present disclosure for a minimally invasive allantoic fluid extraction device not only ensures the hatchability of chicken embryos but also enables rapid collection. During the early stages of egg incubation, this method combines liquid chromatography-tandem mass spectrometry (LC-MS/MS) with bioinformatics analysis to confirm the sexual dimorphism of metabolites in allantoic fluid and the sex-specific metabolites of chicken embryos. The concentration and fractionation strategy designed by this method effectively enriches the low-molecular-weight substances in the allantoic fluid, constructing a multi-component, multi-machine learning spectral sex classification model. The results demonstrate the contribution of the fingerprint spectral characteristic data of low-molecular-weight molecules in the allantoic fluid to sex classification, achieving rapid classification of the sex of chicken embryos.

A system for detecting the sex of the hatching eggs in the early stage of incubation of the present disclosure includes:

A data acquisition unit, configured to confirm the allantoic fluid containing the sex-specific metabolites, obtain the fingerprint spectral characteristic data of the allantoic fluid containing the sex-specific metabolites and add the sex labels, thereby constructing the training set;

A model training unit, configured to construct the multiple machine learning models and train the models through the training set, so as to obtain a trained multi-machine learning allantoic fluid sex classification model;

A sex detection unit, configured to detect the fingerprint spectral characteristic data of the allantoic fluid containing the sex-specific metabolites using the multi-machine learning allantoic fluid sex classification model, and to obtain and present the sex of the hatching eggs in the early stage of incubation on a display.

An electronic device of the present disclosure includes: a memory and a processor coupled to each other, wherein the memory stores program data, and the processor invokes the program data to perform the method as described above.

A computer-readable storage medium of the present disclosure has program data stored therein, wherein when the program data is executed by a processor, the method as described above is realized.

The advantageous effects of the present disclosure are:

The present disclosure for the first time identifies L-ornithine, proline, and cytidine in allantoic fluid as sexually dimorphic biomarkers, with an Area Under the Curve (AUC) value reaching 0.87. These findings were validated through in vitro gonadal cell culture experiments, demonstrating their role in regulating the expression of sex-related genes, thereby providing a molecular basis for sex determination.

The present disclosure, through the integration of allantoic fluid metabolomics and infrared fingerprint spectroscopy analysis, systematically reveals sex-specific metabolite characteristics. The construction of sex-specific spectral fingerprints compensates for the shortcomings of metabolomics in analyzing the global chemical composition.

The present disclosure integrates a concentration and classification method, spectroscopic technology, and machine learning to construct a sex classification model for multi-component allantoic fluid. The present disclosure demonstrates the effectiveness of fingerprint spectral characteristic data of low-molecular-weight substances in allantoic fluid for sex classification, facilitating rapid sex detection in the early stages of incubation of chick embryos.

The present disclosure proposes a comprehensive process of “early minimally invasive sampling-differentiated metabolite screening-low-molecular-weight component enrichment-multispectral machine learning,” achieving breakthroughs in multiple dimensions such as time window, accuracy, and cost control for avian egg sex detection.

### BRIEF DESCRIPTION OF THE DRAWINGS

FIG. 1 is a flowchart of a method for detecting sex of hatching eggs of the present disclosure.

FIG. 2 is a schematic diagram of a minimally invasive extraction device of the present disclosure.

FIG. 3 is a diagram of results of metabolomic classification of allantoic fluid of the present disclosure.

FIG. 4 is a diagram of a sex-specific metabolite module and a receiver operating characteristic curve (ROC) of the present disclosure.

FIG. 5 is a result diagram of a multi-machine learning sex classification model of the present disclosure.

### DESCRIPTION OF THE EMBODIMENTS

The present disclosure is further described in detail below with reference to the accompanying drawings and specific embodiments.

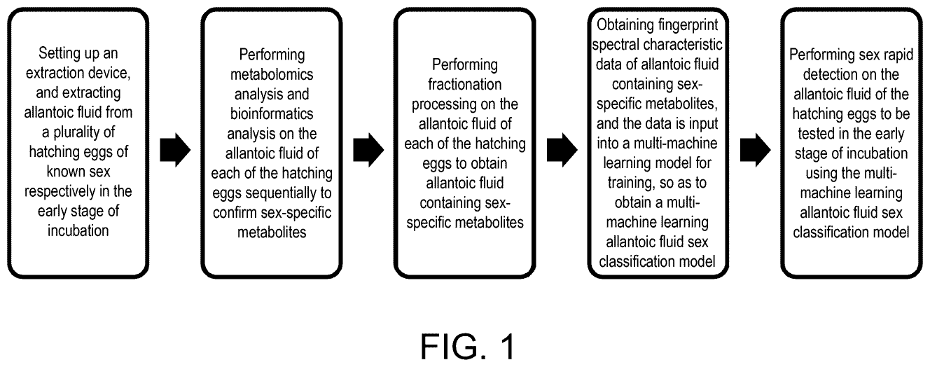

As shown in FIG. 1, a method for detecting sex of hatching eggs in an early stage of incubation of the present disclosure is specifically as follows:

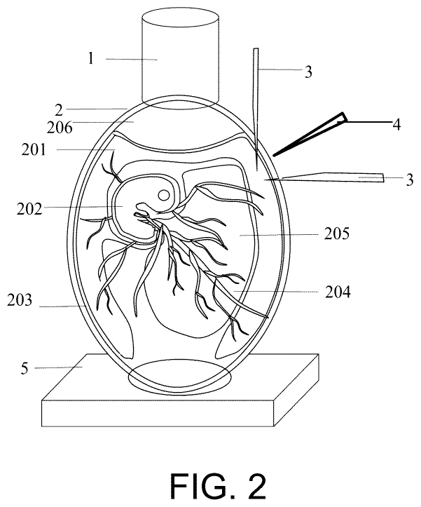

First, an extraction device as illustrated in FIG. 2 is constructed. The extraction device includes a light source 1, an extractor 3, a laser perforator 4 and an egg tray 5. After cleaning the hatching eggs 2 in the early stage of incubation, the hatching eggs 2 are placed vertically on the egg tray 5. The light source 1 is placed on eggshells 203 of top surfaces of the hatching eggs 2 in the early stage of incubation and faces the interior of the hatching eggs 2 in the early stage of incubation downward vertically to visualize positions of the allantoic fluid 201, chicken embryos 202, blood vessels 204, yolk 205 and air cells 206 inside the hatching eggs 2 in the early stage of incubation, so as to avoid other parts and facilitate the extraction of the allantoic fluid 201. During the extraction of the allantoic fluid, the hatching eggs 2 in the early stage of incubation are in a dark environment. The position of the allantoic fluid 201 of the hatching eggs 2 in the early stage of incubation is visualized through the light source 1, then laser perforation is performed on the eggshell 203 of the hatching eggs 2 in the early stage of incubation through the laser perforator 4 to connect to the position of the allantoic fluid 201, an angular range of the extractor 3 is controlled within a range from 30° to 120° on a microporous sectional plane, and finally the allantoic fluid 201 is extracted from the hatching eggs 2 through the extractor 3. Finally, liquid wax is further utilized to seal a micropore to ensure normal incubation of the chicken embryos 202. The extraction of the allantoic fluid 201 from several hatching eggs 2 of known sex is performed separately using the extraction device during the early stage of incubation, specifically between the 5th and 10th day of incubation of the hatching eggs.

Subsequent to conducting metabolomics analysis and bioinformatics analysis on the allantoic fluid 201 of each of the hatching eggs 2, sex-specific metabolites are identified. The metabolomics analysis of the allantoic fluid 201 of each of the hatching eggs 2 is performed using liquid chromatography-tandem mass spectrometry (LC-MS/MS) to identify various metabolites in the allantoic fluid 201 of the hatching eggs 2 with known sex. Thereafter, bioinformatics analysis methods are applied to the metabolites in the allantoic fluid 201 to select for sex-specific metabolites. The metabolomics analysis is employed to compare the metabolites in the allantoic fluid 201 of male and female chicken embryos 202. Subsequently, machine learning and weighted gene co-expression network analysis methods are utilized to identify modules of sex-specific metabolites. The module of sex-specific metabolites includes L-ornithine, proline, and cytidine, among others. This module is used for sex classification of chicken embryos 202, achieving an area under a curve (AUC) value of 0.87.

The reliability of sex-specific metabolites is validated through in vitro culture of gonadal cells. This validation involves administering different concentrations of L-ornithine, proline, and cytidine to interfere with the gonadal and mesonephric cells of the chicken embryos 202, followed by in vitro culture for 24 to 48 hours. The expression of sex-specific genes is then detected using real-time quantitative Polymerase Chain Reaction (PCR) to ascertain the effectiveness of the sex-specific metabolites. These metabolites include L-ornithine, proline, and cytidine, among others.

The allantoic fluid 201 from each of the hatching eggs 2 undergoes a process of concentration and fractionation. Initially, the allantoic fluid 201 from the hatching eggs 2 to be detected is concentrated, which may include evaporation concentration, ultrafiltration concentration, dialysis concentration, among others. The fluid is then fractionated based on molecular weight to obtain high-molecular-weight, medium-molecular-weight, and low-molecular-weight allantoic fluids, specifically isolating the allantoic fluid 201 that contains sex-specific metabolites, with the low-molecular-weight allantoic fluid including these sex-specific metabolites, thus enriching the metabolite information of the low-molecular-weight allantoic fluid. The high-molecular-weight allantoic fluid is the allantoic fluid 201 characterized by a molecular weight greater than 30 KDa, the medium-molecular-weight allantoic fluid is the allantoic fluid 201 having a molecular weight between 30 KDa and 10 KDa, and the low-molecular-weight allantoic fluid is the allantoic fluid 201 having a molecular weight below 10 KDa. The method of concentration and fractionation of the allantoic fluid 201 involves an ultrafiltration concentration and fractionation strategy, with the pre-concentration of various components of the allantoic fluid 201 onto the surface of a crystal or slide to determine optimal spectral parameters for acquiring a fingerprint spectrum. Specifically, an infrared spectral fingerprint characteristic peak at 1650 cm⁻¹ of the allantoic fluid 201 is attributed to the amide I anti-parallel β-helix, indicating protein CO stretching vibration. Lipid-specific bands include a band at 1454 cm⁻¹, corresponding to CH₂ bending in lipid acyl chains, and at 1390 cm⁻¹, representing COO⁻ symmetric stretching in fatty acids and amino acids. The band at 1210 cm⁻¹ is related to PO₂-antisymmetric stretching in phospholipids and nucleic acids, and the band at 1040 cm⁻¹ originates from PO₂-symmetric stretching in dianionic phosphate monoesters found in phosphorylated proteins and nucleic acids. The spectral region spanning 675 cm⁻¹ to 900 cm⁻¹ is the most prominent area for aromatic compounds. Spectral changes observed at approximately 918 cm⁻¹, 845 cm⁻¹, 676 cm⁻¹, and 486 cm⁻¹ may be attributed to out-of-plane bending vibrations of CH groups in substituted benzene rings, indicating aromatic compounds. In summary, the achieved classification accuracy may be attributed to changes in protein conformation and lipid intensity, as well as features of amino acids and nucleic acids.

Subsequently, the allantoic fluid 201, containing sex-specific metabolites from each of the hatching eggs 2, is collected after enrichment on the surface of a crystal or slide for spectral scanning. Spectral techniques, including infrared spectroscopy and Raman spectroscopy, are then used to obtain the fingerprint spectral characteristic data of the allantoic fluid 201. Infrared spectroscopy provides in-depth insights into molecular vibrations, rotations, and stretching. The allantoic fluid concentration and classification strategy enriches low-molecular-weight substances on the surface of the crystal, facilitating a deeper understanding of absorption peaks specific to AF metabolites of the chicken embryos. In specific implementations, the differentiation regions in infrared spectroscopy show correlation with the classification of identified metabolites. For example, protein-related metabolites: in analyses on incubation days D8 and D9, amino acids such as proline, L-ornithine, and L-phenylalanine significantly increased in males. The protein structural characteristics of these amino acids may be reflected by the amide peak at 1650 cm⁻¹, indicating changes in protein conformation. Lipid-related metabolites: in the analysis on incubation day D9, 3-hydroxybutyric acid and D-(+)-malic acid significantly increased in males. The characteristics of these lipid metabolites may be reflected by the lipid characteristic peaks at 1454 cm⁻¹ and 1390 cm⁻¹, indicating sex differences in lipid metabolism. Nucleic acid-related metabolites: in the analysis on day D10, uridine and cytidine significantly increased in females. The characteristics of these nucleic acid metabolites may be reflected by the PO₂-stretching vibration peaks at 1210 cm⁻¹ and 1040 cm⁻¹, indicating sex differences in nucleic acid metabolism.

Subsequently, after adding labels of known sex to the fingerprint spectral characteristic data, a training set is constructed. This training set is then input into multiple machine learning models for training, testing, and validation, resulting in a trained multiple machine learning models for allantoic fluid sex classification.

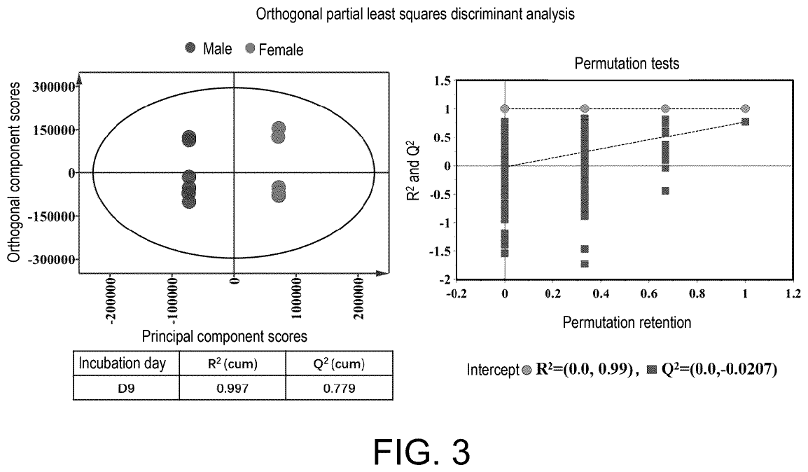

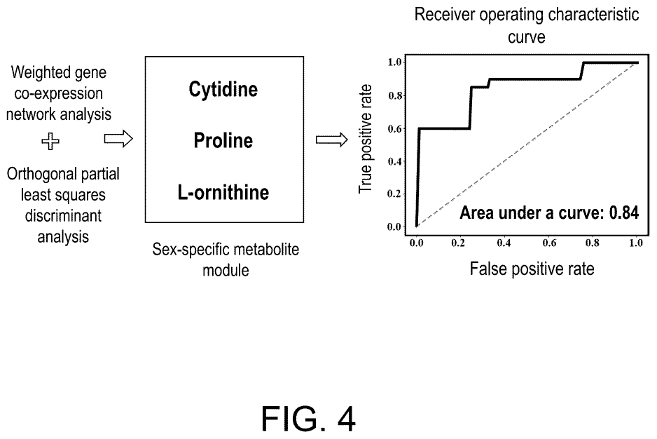

The present disclosure, in its specific implementation, was based on non-targeted metabolomics to study the differential metabolites in the allantoic fluid 201 of male and female chicken embryos 202. The allantoic fluid 201 extracted from Jinghong No. 1 hatching eggs 2, under actual breeding conditions, was selected as the test subject for the implementation example. Initially, liquid chromatography coupled with tandem mass spectrometry (LC-MS/MS) was employed to analyze the metabolites in the allantoic fluid 201 of the chicken embryos 202 at different incubation days. The classification results of Orthogonal Partial Least Squares Discriminant Analysis (OPLS-DA) are shown in FIG. 3, from which orthogonal component scores and principal component scores for both male and female may be obtained. On the 9th day of incubation, an explanatory parameter R²=0.997 and a predictive parameter Q²=0.779, indicating that the metabolites in the allantoic fluid 201 of the chicken embryos 202 exhibit sexual dimorphism. Subsequently, after 200 permutation tests, results for permutation retention, as well as explanatory and predictive parameters, were obtained, with an explanatory parameter R²=0.99 and a predictive parameter Q²=−0.0207, validating the reliability of the model and ensuring no overfitting. A machine learning dataset was constructed by combining metabolites selected through Weighted Gene Co-expression Network Analysis and OPLS-DA. The characteristic importance assessment results of the metabolites are shown in FIG. 4, with higher contributing metabolites, such as L-ornithine, proline, and cytidine, chosen as sex-specific metabolites. The results of using sex-specific metabolites for sex classification indicated an area under the Receiver Operating Characteristic (ROC) curve of 0.84.

Subsequently, the validity of sex-specific metabolites was verified using in vitro cell culture. Initially, the gonadal and mesonephric tissues of the chicken embryos 202 were extracted and cultured under sterile conditions. The gonadal cells were treated with candidate sex-specific metabolites at varying concentration gradients. An optimal metabolite concentration for cell proliferation was determined to be 200 μM using the Cell Counting Kit-8 (CCK8).

Based on real-time quantitative polymerase chain reaction (PCR) analysis, the differences in sex marker gene expression between gonadal cells treated with 200 μM of L-ornithine, proline, and cytidine and untreated control cells were examined. The results indicate that treatment with cytidine (test result parameter P<0.001) and L-ornithine (test result parameter P<0.001) significantly reduced the expression of anti-Müllerian hormone (AMH) in the gonadal cells of both male and female chicken embryos 202, further increasing the difference in AMH expression between the sexs. Cytidine treatment (test result parameter P<0.01) significantly downregulated the expression of DMRT1 (Double sex and mab-3 related transcription factor 1) in the gonads of male chicken embryos 202, while proline (test result parameter P<0.05) induced upregulation, further increasing the difference in DMRT1 expression between males and females. In the gonadal cells of female chicken embryos 202, both cytidine (test result parameter P<0.01) and L-ornithine (test result parameter P<0.01) significantly downregulated CYP19A1 (cytochrome P450, family 19, subfamily A, polypeptide 1) expression, while proline upregulated its expression in male cells. The treatments with cytidine and proline reduced the existing differences in CYP19A1 expression between the sexs. Similarly, cytidine (test result parameter P<0.01) and L-ornithine (test result parameter P<0.01) significantly downregulated FOXL2 (forkhead box L2) expression in female gonads, whereas proline upregulated its expression in males. These treatments also reduced the differences in FOXL2 expression between the sexs.

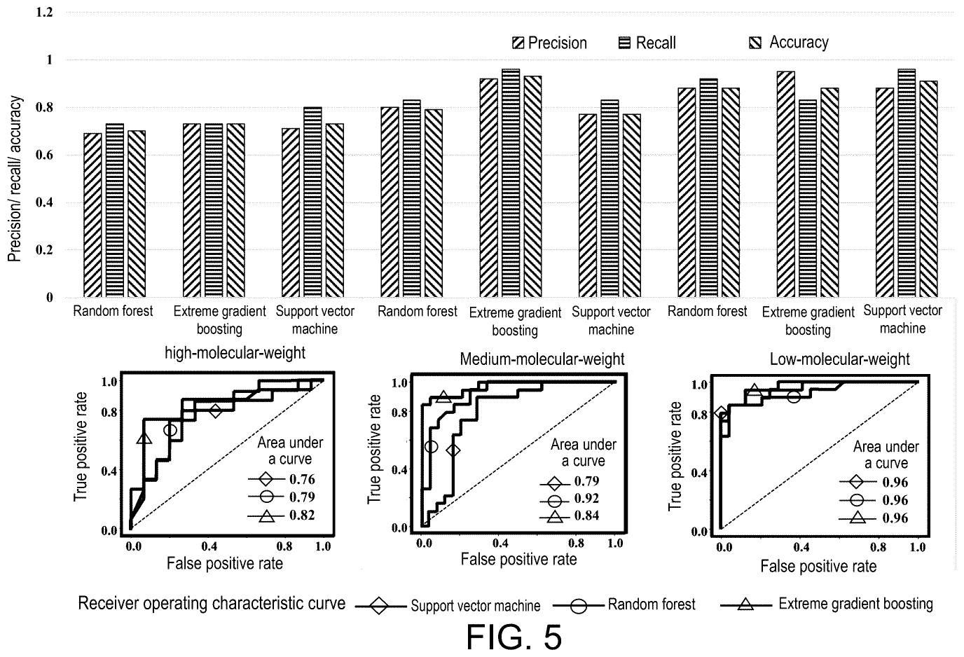

As illustrated in FIG. 5, the present disclosure constructs a spectral classification model based on fingerprint spectral data of multi-component allantoic fluid using various machine learning techniques: Random Forest, Support Vector Machine (SVM), and Extreme Gradient Boosting. The in-depth training employs five parameters, including the area under the Receiver Operating Characteristic (ROC) curve, classification accuracy, precision, recall, and the ROC curve itself, to comprehensively evaluate the classification performance of the spectra. Specifically, the allantoic fluid 201 of the male chicken embryos 202 is used as the experimental group, while the female serves as the control group. The results of the SVM classification of the sex of the chicken embryos 202 under different concentration grading parameters are as follows: 1) Low-molecular-weight: precision: 88%, recall: 92%, accuracy: 88%, area under the ROC curve (AUC): 0.96; 2) Medium-molecular-weight: precision: 78%, recall: 83%, accuracy: 80%, AUC: 0.92; 3) High-molecular-weight: precision: 70%, recall: 73%, accuracy: 68%, AUC: 0.79. The results indicate that high-molecular-weight substances in the allantoic fluid 201 may obscure the spectral signals of low-molecular-weight substances, thereby limiting their contribution to sex classification spectra. When only the spectral signals of low-molecular-weight substances in the allantoic fluid 201 are considered, the spectral classification results are significantly improved. The research findings of the present disclosure emphasize that the spectral classification results of low-molecular-weight allantoic fluid 201 after ultrafiltration concentration are superior to those of untreated and high-molecular-weight allantoic fluid 201.

The allantoic fluid 201 from the hatching eggs 2 in the early stage of incubation is ultimately extracted using the extraction device. The fingerprint spectral characteristic data is then obtained and input into multiple machine learning models for allantoic fluid sex classification. Through this process, the sex of the hatching eggs 2 in the early stage of incubation is determined, thereby enabling rapid sex detection of the hatching eggs 2 in the early stage of incubation.

The use of non-targeted metabolomics combined with bioinformatics analysis has demonstrated the sexual dimorphism of metabolites in the allantoic fluid 201 of the chicken embryos 202, providing a reference for the feasibility of using low-molecular-weight substances for sex classification. In vitro cell culture experiments have verified the significant impact of sex-specific metabolites on sex marker genes. Given the complexity of the allantoic fluid 201 samples, a concentration and fractionation strategy designed during the early incubation process may effectively simplify complex mixtures like the allantoic fluid 201, enriching low-molecular-weight substances and ensuring the contribution of characteristic spectra of low-molecular-weight substances in the allantoic fluid 201. The present disclosure achieves high-precision, non-destructive detection of sex of the hatching eggs in the early stage through novel minimally invasive sampling, sex-specific metabolite screening, and multi-modal data analysis techniques. The present disclosure carries both scientific value and industrial application prospects, providing a technical solution to enhance the efficiency of the poultry industry and address ethical controversies such as the culling of male chicks.

The present disclosure also establishes a system for detecting sex of hatching eggs in the early stage of incubation, including a data acquisition unit, a model training unit, and a sex detection unit. The data acquisition unit is designed to identify the allantoic fluid 201 that contains the sex-specific metabolites, acquire the fingerprint spectral characteristic data of such allantoic fluid 201, and add sex labels, thereby constructing the training set. The model training unit is employed to build multiple machine learning models and train them using the training set to obtain a fully trained multi-machine learning allantoic fluid sex classification model. The sex detection unit utilizes the multi-machine learning allantoic fluid sex classification model to detect the fingerprint spectral characteristic data of the allantoic fluid 201, which contains the sex-specific metabolites and is subjected to an analysis, then the sex of the hatching eggs 2 to be detected in the early stage of incubation are determined and presented on a display.

The present disclosure discloses a method revealing the sexual dimorphism of metabolites in the allantoic fluid. Accordingly, a designed concentration and grading method, combined with multi-machine learning, demonstrates the contribution of the spectral characteristics of low-molecular-weight substances in the allantoic fluid to sex classification. This research method may also provide new insights into the development of avian egg sex identification technology.

Professionals in the relevant field should understand that the embodiments of the present disclosure may be provided as a method, system, or computer program product. Consequently, the present disclosure may be implemented as a fully hardware-based embodiment, a fully software-based embodiment, or an embodiment that combines both software and hardware aspects. Furthermore, the present disclosure may be implemented in the form of a computer program product on one or more computer-usable storage media containing computer-usable program code, including but not limited to magnetic storage, optical storage, and the like. The solutions in the embodiments of the present disclosure may be implemented using various computer languages. The present disclosure is described with reference to flowcharts of the methods, systems, and computer program products according to the embodiments of the present disclosure.

Although the preferred embodiments of the present disclosure have been described, those skilled in the art, once apprised of the fundamental inventive concept, may make further changes and modifications to these embodiments. Therefore, the disclosure is intended to be construed as including the preferred embodiments as well as all changes and modifications that fall within the scope of the present disclosure.

It is apparent that those skilled in the art may make various modifications and variations to the present disclosure without departing from the spirit and scope thereof. Thus, if such modifications and variations fall within the scope of equivalent technologies of the present disclosure, the present disclosure is intended to encompass such modifications and variations.

What is claimed is:

1. A method for detecting sex of hatching eggs in an early stage of incubation, comprising:
step 1: setting up an extraction device, and extracting allantoic fluid from a plurality of hatching eggs of known sex respectively in the early stage of incubation through the extraction device;
step 2: performing a metabolomics analysis and a bioinformatics analysis on the allantoic fluid of each of the hatching eggs sequentially to confirm sex-specific metabolites;
step 3: performing fractionation processing on the allantoic fluid of each of the hatching eggs to obtain the allantoic fluid containing the sex-specific metabolites;
step 4: obtaining fingerprint spectral characteristic data of the allantoic fluid containing the sex-specific metabolites from each of the hatching eggs and adding labels of the respective known sex, thereby constructing a training set to input into multiple machine learning models for training, so as to obtain a trained multi-machine learning allantoic fluid sex classification model;
step 5: extracting the allantoic fluid of the hatching eggs to be detected in the early stage of incubation through the extraction device, then obtaining the fingerprint spectral characteristic data of the allantoic fluid containing the sex-specific metabolites to be detected after performing the same processing as in steps 3 and 4, and inputting the fingerprint spectral characteristic data into the multi-machine learning allantoic fluid sex classification model, obtaining the sex of the hatching eggs in the early stage of incubation to be detected after processing, thus achieving rapid sex detection of the hatching eggs in the early stage of incubation;
wherein in the step 2, the sex-specific metabolites comprise L-ornithine, proline and cytidine.

2. The method for detecting the sex of the hatching eggs in the early stage of incubation according to claim 1, wherein in step 1, the extraction device comprises a light source, an extractor, a laser perforator and an egg tray, the hatching eggs in the early stage of incubation are placed with the blunt end facing upward, the light source is placed on the top surface of the eggshells of the hatching eggs in the early stage of incubation and faces the interior of the hatching eggs in the early stage of incubation downward vertically, during extraction of the allantoic fluid, the hatching eggs in the early stage of incubation are in a dark environment, a position of the allantoic fluid of the hatching eggs in the early stage of incubation is visualized by means of the light source, then laser perforation is performed on the eggshells of the hatching eggs in the early stage of incubation through the laser perforator to connect to the position of the allantoic fluid, and finally the allantoic fluid is extracted from the hatching eggs through the extractor.

3. The method for detecting the sex of the hatching eggs in the early stage of incubation according to claim 1, wherein in step 1, the early stage of incubation specifically refers to a period of 5 to 10 days of incubating the hatching eggs.

4. The method for detecting the sex of hatching eggs in the early stage of incubation according to claim 1, wherein in step 2, the metabolomics analysis is performed on the allantoic fluid of each of the hatching eggs by: firstly, identifying respective metabolites present in the allantoic fluid of hatching eggs of known sex using Liquid Chromatography-Tandem Mass Spectrometry (LC-MS/MS); and subsequently, employing a bioinformatics analysis method to screen for sex-specific metabolites from the metabolites identified in the allantoic fluid corresponding to each of the known sex.

5. The method for detecting the sex of the hatching eggs in the early stage of incubation according to claim 1, wherein in the step 3, the allantoic fluid of the hatching eggs to be detected is first concentrated and then fractionated according to molecular-weight to obtain high-molecular-weight allantoic fluid, medium-molecular-weight allantoic fluid, and low-molecular-weight allantoic fluid, the low-molecular-weight allantoic fluid is the allantoic fluid containing the sex-specific metabolites.

6. The method for detecting the sex of the hatching eggs in the early stage of incubation according to claim 1, wherein in the step 4, for each of the hatching eggs, the allantoic fluid containing the sex-specific metabolites is enriched on a surface of a crystal or a slide, subsequently, a spectral scan is conducted to collect data, thereby obtaining the fingerprint spectral characteristic data of the allantoic fluid.

7. A system for detecting sex of hatching eggs in an early stage of incubation for the method according to claim 1, comprising:
a data acquisition unit, configured to confirm the allantoic fluid containing the sex-specific metabolites, obtaining fingerprint spectral characteristic data of the allantoic fluid containing the sex-specific metabolites and adding sex labels, thereby constructing a training set;
a model training unit, configured to construct the multiple machine learning models and train the model through the training set, so as to obtain a trained multi-machine learning allantoic fluid sex classification model;
a sex detection unit, configured to detect the fingerprint spectral characteristic data of the allantoic fluid containing the sex-specific metabolites to be detected through the multi-machine learning allantoic fluid sex classification model, then the sex of the hatching eggs to be detected in the early stage of incubation are obtained and presented on a display.

8. An electronic device, comprising: a memory and a processor coupled to each other, wherein the memory stores program data, and the processor invokes the program data to execute the method according to claim 1.

9. A computer-readable storage medium having program data stored therein, wherein, when the program data is executed by a processor, the method according to claim 1 is realized.
